(19) United States
(12) Patent Application Publication
(10) Pub. No.: US 2007/0213612 A1
(43) Pub. Date: Sep. 13, 2007

(54) Method of urodynamic diagnostics

(76) Inventors: Yitzhak Rosen, Reston, VA (US); Oleg Shapiro, Tel Aviv (IL)

Correspondence Address: Dr. Yitzhak Rosen, Apt. A4, 12003 Greywing Square, Reston, VA, 20191-1870, US

(21) Appl. No.: 11/371,276

(22) Filed: Mar. 9, 2006

(51) Int. Cl. A61B 8/00

(52) U.S. Cl. 600/438

(57) ABSTRACT

The suggested method of urodynamic diagnostics envisages the assessment of the presence of detrusor activity. The method characterizes detrusor activity caused by a condition of the urinary system not influenced by provocative factors. The said detrusor activity is being detected non-invasively. That is, true detrusor activity is detected by means of ultrasound examination. The method is intended for the diagnostic assessment of involuntary urination and for diagnostic assessment of impaired urination. The method allows an increased accuracy of urodynamic diagnostic and assessment detrusor activity, non-invasively.

### BACKGROUND OF THE INVENTION

1. Field of the Invention

The present invention relates to the field of urology, and, more particularly, to methods of urodynamic diagnostics.

2. State of the Art

It is widely known that urodynamic diagnostics are performed to assess the condition of the urinary system, in particular, to identify diseases related to hyper- or hypoactivity of the urinary bladder.

One of the main parameters assessed in urodiagnostics is stability or instability functionality of detrusor activity (vibration of the urinary bladder wall).

An invasive method of urodynamic diagnostics envisaging insertion of a catheter into the urinary bladder via the urethra is known and widely used. Together with determining the volume of the urinary bladder and/or the difference of pressures in the bladder and in the urethra, the diagnostic procedure also includes detection of pressure fluctuations in the urinary bladder. The said fluctuations taking place, detrusor activity is assumed to be present. Depending on the said parameters one or another disease is diagnosed.

According to this method, detrusor activity is detected indirectly by the said fluctuations of pressure in the urinary bladder.

The indirect detection of detrusor activity either considerably reduces the diagnostic accuracy, or requires the use of additional diagnostic means making the diagnostic procedure more complex. This is accounted for by the following reasons:
1. It is known that fluctuations of pressure in the urinary bladder may be brought about not only by detrusor activity but by some foreign provocative factors such as fluctuations of pressure in the abdominal cavity, the patient's involuntary movements in the course of diagnostics, and others. That is why this method does not make it possible to identify the true detrusor activity, i.e., detrusor activity caused by a condition of the urinary system rather than by a provocative factor. To eliminate the effect of the said provocative factors on fluctuations of pressure in the urinary bladder, additional means are required which make the diagnostics more complicated.
2. Fluctuations of pressure in the urinary bladder, under some frequencies and/or amplitudes of vibration of its wall, are so weak that they are undetectable.

Another important drawback of this method consists in mechanical irritation of the urinary bladder wall by a catheter inserted into the bladder, the said irritation sometimes causing or suppressing detrusor activity. This fact, too, impairs the diagnostic accuracy.

Besides, insertion of a catheter is painful for the patient and may even cause an inflammation in the urinary bladder or the urethra. Therefore, this diagnostic method is exceptionally rarely used in children.

The probability of inadequately accurate diagnostics with a catheter is also confirmed by academic literature (“Campbell's urology” W. B. Saunders; 8^{th} edition, Jun. 15, 2002, page 909) saying that if no detrusor activity has been detected a diagnosis of urge incontinence strictly conditional upon presence of detrusor activity still cannot be excluded.

### SUMMARY OF THE INVENTION

The object of the present invention is, therefore, increasing the accuracy of urodynamic diagnostics.

Another object of the present invention is sparing pain sensations to the patients in the course of diagnostics.

Still another object of the present invention consists in prevention of inflammatory processes in the urinary bladder and the urethra that may be brought about by the conventional diagnostic methods.

And, lastly, one more object of the present invention is simplifying the diagnostic procedure by dispensing with additional diagnostic means impairing the diagnostic accuracy.

The said and other objects are attained due to the circumstance that it is the true detrusor activity that is only detected in the course of urodynamic diagnostics, i.e. detrusor activity caused by a condition of the urinary system uninfluenced by provocative factors, said detrusor activity being detected non-invasively.

The present invention will be understood more fully from the detailed description given herein below which, however, should not be construed as exhaustive to the invention but is for explanation and understanding only.

### DETAILED DESCRIPTION OF THE PREFERRED EMBODIMENTS OF THE INVENTION

Below the procedure of diagnosing diseases in involuntary or impaired urination by means of ultrasound (US) is described.

In involuntary urination the diagnostics are performed as follows.

Before a diagnostic procedure the patient is made to drink an amount of liquid sufficient to presently fill the urinary bladder. Then, in 20 to 30 minutes, the patient is made to lie onto a couch and the US transducer is placed perpendicular or at another desired angle to a specific site in the relevant perineal or abdominal region. The process of filling of the bladder visualized by US is recorded on video. Detrusor activity is assessed using the tissue Doppler imaging technology or the Dynamic US. As long as the presence of detrusor activity is detected directly (visually), contrary to the catheter method where it is detected indirectly by fluctuations of pressure and the findings are influenced by various provocative factors, the proposed method allows to detect the true detrusor activity caused solely by a condition of the urinary system uninfluenced by any provocative factors. Once the bladder is full, the patient is asked to cough. While the patient is coughing the presence of detrusor activity and involuntary urination is assessed by means of US. With both symptoms present, urge incontinence is diagnosed. When, with involuntary urination present, detrusor activity is absent, stress incontinence is diagnosed.

In the case of impaired urination the patient is asked to urinate after his bladder has been filled. In the course of urination the presence of detrusor activity, the cross-sectional size of the urethra and the urethral flow rate are assessed by means of US. The difference of pressures in the bladder and in the urethra is determined by a known method (by the Bernoulli formula) based on the cross-sectional size of the urethra and the urethral flow rate obtained. If the difference of pressures is above . . . Pa and detrusor activity is present, then outlet obstruction is diagnosed.

In further embodiments of the present invention, instead of ultrasound, other systems may be used based on other principles such as electromagnetic waves i.e., on principles making it possible to assess non-invasively the presence of detrusor activity, the volume of the urinary bladder and the difference of pressures in the bladder and in the urethra.

The present invention makes it possible to increase the accuracy of urodynamic diagnostics, to spare pain sensations to the patients in the course of diagnostics, to prevent inflammatory processes in the urinary bladder and the urethra that may be brought about by the conventional diagnostic methods, and as well to simplify the diagnostic procedure by dispensing with additional diagnostic means impairing the diagnostic accuracy.

Although the invention has been described with a certain degree of particularity, it is understood that the present disclosure has only been made by way of example, and that various modifications thereof may be resorted to by those skilled in the art without departing from the spirit and scope of the invention, as hereinafter claimed.

1. A method of urodynamic diagnostics envisaging assessment of the presence of detrusor activity, according to which it is the true detrusor activity that is only detected, i.e. detrusor activity caused by a condition of the urinary system uninfluenced by provocative factors, said detrusor activity being detected non-invasively.

2. The method according to claim 1, wherein the true detrusor activity is detected by means of ultrasound examination.

3. The method according to claims 1 or 2 intended for diagnostics in involuntary urination, wherein the presence of the true detrusor activity is assessed in the course of involuntary urination, and, with said detrusor activity present, urge incontinence is diagnosed, and with detrusor activity absent, stress incontinence is diagnosed.

4. The method according to claims 1 or 2 intended for diagnostics in impaired urination, wherein the difference of pressures in the urinary bladder and the urethra during urination is assessed, and, with the said difference above . . . and the said true detrusor activity present, outlet obstruction is diagnosed.

5. The method according to claims 4, wherein the said difference of pressures is assessed non-invasively.

6. The method according to any of claims 1 to 5, as described above with reference to the enclosed specification.
